(19) United States
(12) Patent Application Publication
(10) Pub. No.: US 2005/0038395 A1
(43) Pub. Date: Feb. 17, 2005

(54) Retractable safety type operatable with single-hand

(75) Inventor: Chao-Hua Shih, Taipei (TW)

Correspondence Address: TROXELL LAW OFFICE PLLC, SUITE 1404, 5205 LEESBURG PIKE, FALLS CHURCH, VA, 22041, US

(73) Assignee: Life-Shield Products, Inc.

(21) Appl. No.: 10/640,015

(22) Filed: Aug. 14, 2003

(51) Int. Cl. A61M 5/315

(52) U.S. Cl. 604/227

(57) ABSTRACT

A retractable safety type syringe operatable with single-hand comprises: a barrel with a bottom plate extending externally toward all directions on the rear end thereof; a needle set fixedly connected to the front end of the barrel, the needle set having a first engaging portion on the rear end thereof; and a plunger placed in the barrel, having on the front end thereof a second engaging portion to engage with the first engaging portion, and having an end plate on the rear end of the plunger. Thereby, two fingers can be used to hook the bottom plate of the barrel, and another finger of the same hand can be used to push forwards the plunger for injection; after injection, two fingers can be used to abut against the end plate on the rear end of the plunger, another finger of the same hand can be used to push the bottom plate extending out of the barrel, so that the plunger moves relatively to the barrel to get the function of pulling the plunger rearwards. In this mode, the user can push forwards the plunger and can pull back the plunger only with single-hand.

### BACKGROUND OF THE INVENTION

1. Field of the Invention

The present invention is related to a retractable safety type syringe operatable with single-hand; and especially to a retractable safety type syringe convenient for pushing in or drawing back a plunger only with single-hand in drawing or injecting liquid medicine, it suits medical treatment.

2. Description of the Prior Art

In processes of medical treatment, use of syringes is very frequent. Syringes normally are divided into two kinds: a normal type and a retractable type. The normal type has a needle set fixedly connected to the front end of a barrel of a syringe, the needle set is exposed no matter it is before or after injecting, and thereby is quite dangerous; the retractable type has a needle set contracted into a syringe after injecting liquid medicine, and thereby is more reliable in safety and sanitation.

A generally used retractable safety type syringe 1, as shown in FIG. 1, comprises a barrel 2 with two symmetrically arranged wings 3 on the bottom thereof, an injecting needle set 4 connected on the front end of the barrel 2 and a plunger 5 movably frontwards and rearwards in the barrel 2, the plunger 5 has an end plate 6 on the rear end thereof in favor of the operation of pushing and pulling.

In the art presently, in use of a retractable safety type syringe, convenience of using only with single-hand is highly thought of; if it is desired to use the above stated conventional retractable safety type syringe 1 only with single-hand, generally two fingers are used to hook the wings 3 of the barrel 2, and another finger is used to push the plunger 5 to complete injection. After injection, two fingers are used to abut against the end plate 6 on the rear end of the plunger 5, while another finger is used to push the wings 3 extending out of the barrel 2, the plunger 5 moves relatively to the barrel 2 to get the function of pulling the plunger 5 rearwards. By virtue that the ends of the wings 3 extend outwardly to form an elongate shape, one finger must accurately abut on one of the wings 3 when it is to push with the finger; for this sake, sometimes the barrel 2 has to be rotated for an angle. Hence in operating the conventional retractable safety type syringe 1 only with single-hand, it is quite inconvenient by the limitation of the direction of the wings 3 of the barrel 2.

In view of these, the inventor developed the present invention to get rid of the above stated flaws based on his experience of years to allow drawing or injection of liquid medicine to be easily performed only with single-hand.

### SUMMARY OF THE INVENTION

The primary object of the present invention is to provide a retractable safety type syringe operatable with single-hand, the rear end of its barrel is provided with a bottom plate extending externally toward all directions in favor of operating by single hand of a user for pushing in or drawing back a plunger in drawing or injecting liquid medicine.

To achieve the above objects, the retractable safety type syringe of the present invention is comprised of: a barrel with a bottom plate extending externally toward all directions on the rear end thereof; a needle set fixedly connected to the front end of the barrel of the syringe, the needle set having a first engaging portion on the rear end thereof; and a plunger placed in the barrel, having on the front end thereof a second engaging portion to engage with the first engaging portion, and having an end plate on the rear end of the plunger. Thereby, two fingers can be used to hook the bottom plate of the barrel, and another finger can be used to push forwards the plunger to complete injection; and after injection, two fingers can be used to abut against the end plate on the rear end of the plunger, while another finger can be used to push the bottom plate extending out of the barrel, so that the plunger moves relatively to the barrel to get the function of pulling the plunger rearwards. In this mode, the user can push forwards and pull back the plunger only with single-hand.

The present invention will be apparent after reading the detailed description of the preferred embodiments thereof in reference to the accompanying drawings.

### BRIEF DESCRIPTION OF THE DRAWINGS

FIG. 1 is a perspective view of a conventional safety syringe;

FIG. 2 is a perspective view of a first embodiment of the present invention;

FIG. 3 is an analytic perspective view of the first embodiment of the present invention;

FIG. 4 is a sectional view showing assembling of the first embodiment of the present invention;

FIG. 5 is a schematic perspective view showing use of the present invention in injection;

FIG. 6 is a schematic perspective view showing use of the present invention in drawing out a plunger;

FIG. 7 is a sectional view after assembling of the of the present invention in drawing out a plunger;

FIG. 8 is a perspective view of a second embodiment of the present invention;

FIG. 9 is a perspective view of a third embodiment of the present invention;

FIG. 10 is a sectional view after assembling of another embodiment of the present invention.

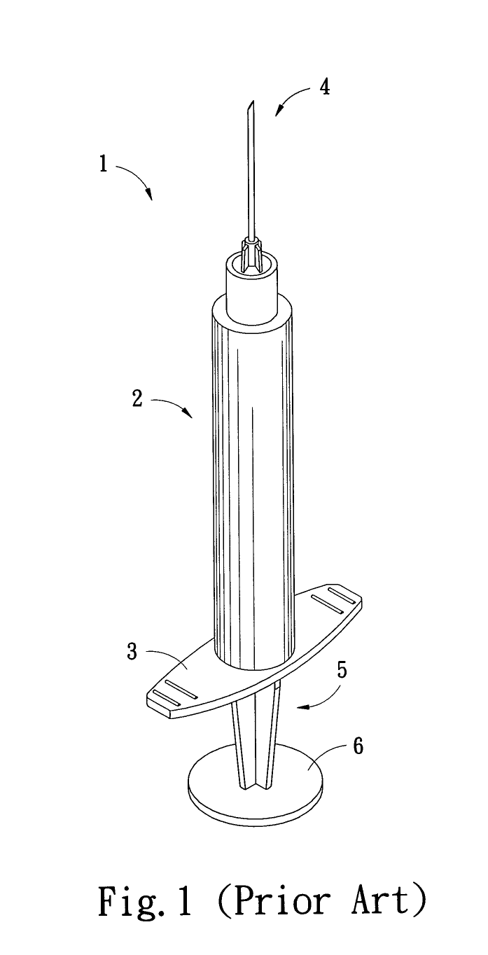

### DETAILED DESCRIPTION OF THE PREFERRED EMBODIMENT

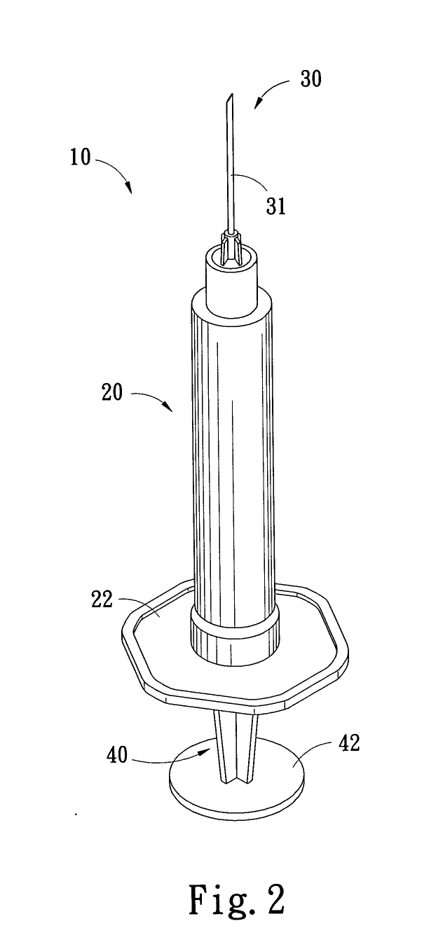

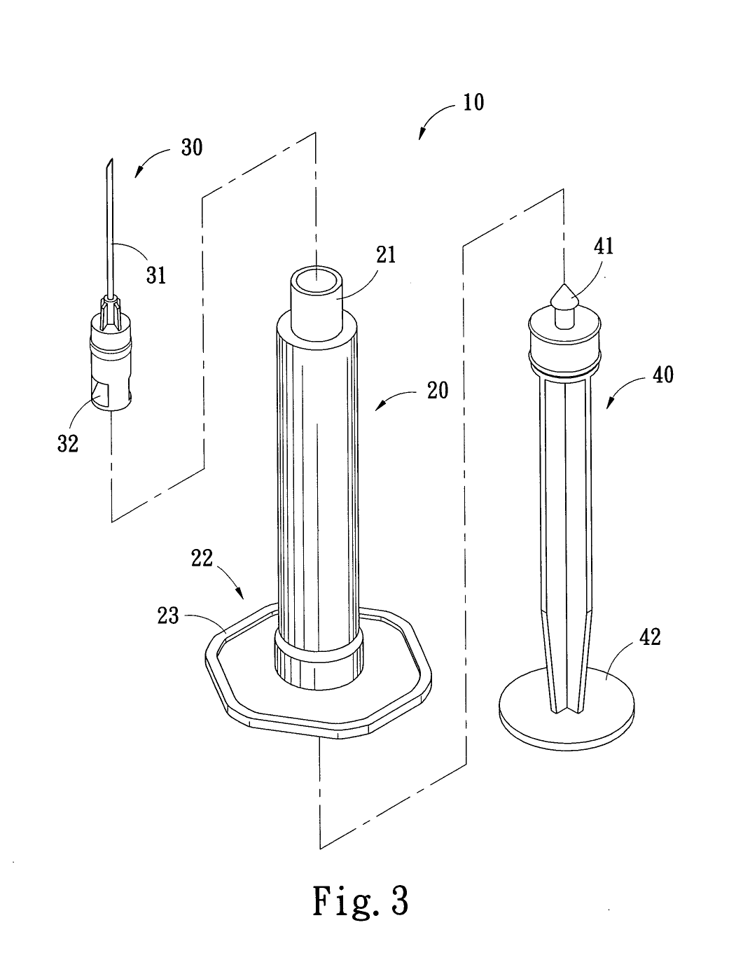

Referring to FIGS. 2 and 3 showing a first embodiment of a retractable safety type syringe 10 operatable with single-hand of the present invention, it comprises mainly: a barrel 20, a needle set 30 and a plunger 40, wherein:

The barrel 20 is a hollow cylinder, the inner wall of its front end 21 can be used to connect thereto the needle set 30, and its rear end is provided with a bottom plate 22 extending externally toward all directions and perpendicular to the axis of the hollow cylinder, the bottom plate 22 is a hexagonal plate.

The needle set 30 is connected to the inner wall on the front end 21 of the barrel 20, and has a hollow injection needle 31 on the front end of itself, and further has a first engaging portion 32 on the rear end thereof.

The plunger 40 can be placed in the barrel 20, it has on the front end thereof a second engaging portion 41 to engage with the first engaging portion 32, and has an end plate 42 on the rear end thereof extending externally and perpendicular to the axis thereof.

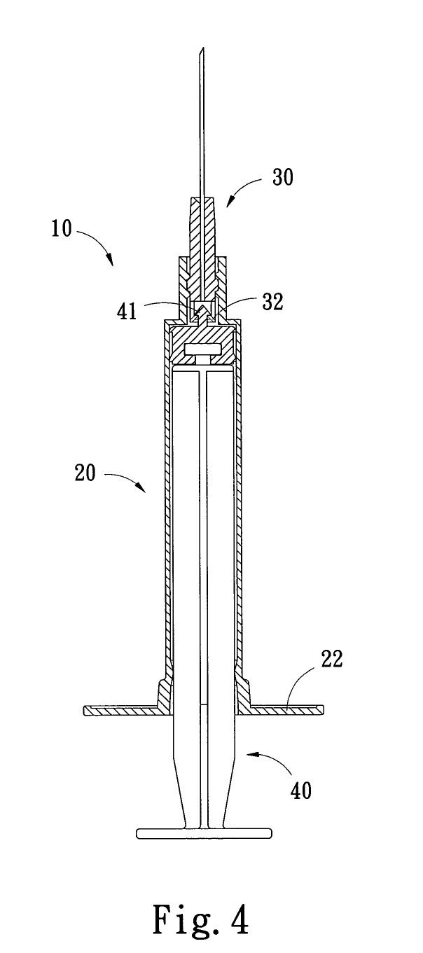

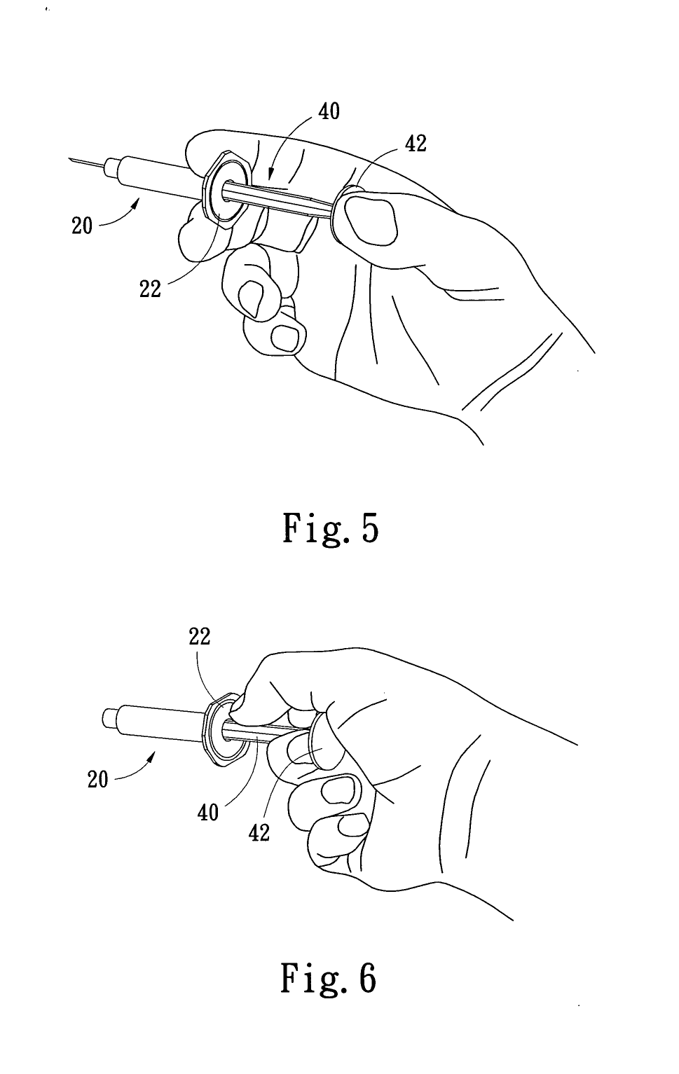

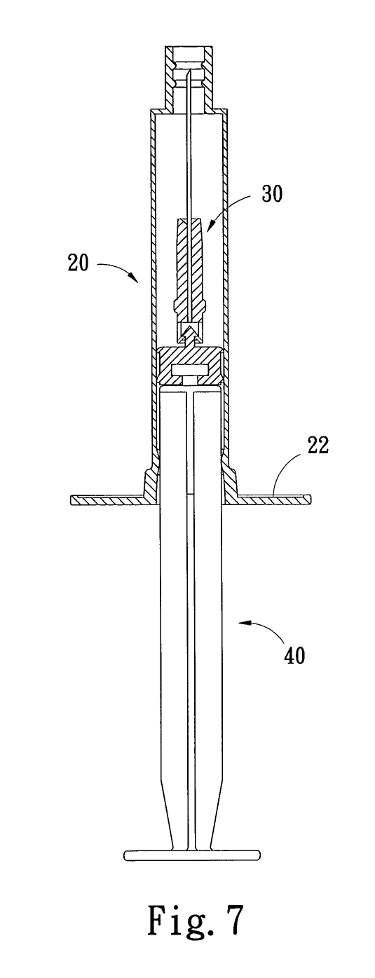

Referring to FIGS. 4-7, in operation of the present invention, an index finger and a middle finger can be used to hook the bottom plate 22 of the barrel 20, and another finger can be used to abut against the rear end plate 42 of the plunger 40, then a thumb is used to push the bottom plate 22 extending out of the barrel 20, so that the plunger 40 moves relatively to the barrel 20 to get the function of pulling the plunger 40 rearwards.

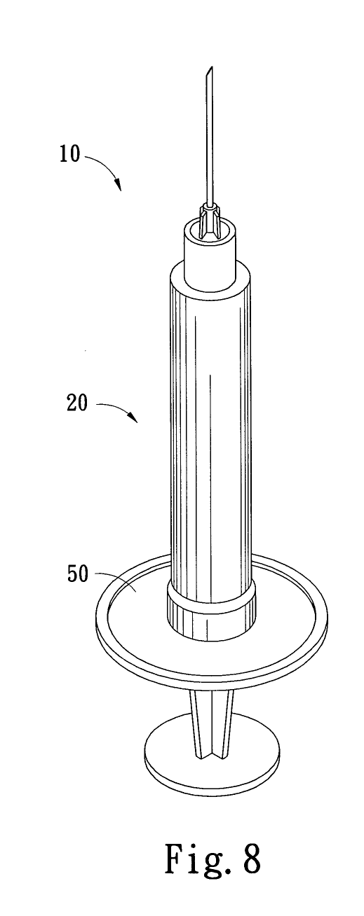

Referring to FIG. 8 showing a second embodiment of the retractable safety type syringe 10 operatable with single-hand of the present invention, wherein a bottom plate 50 on the rear end of the barrel 20 is a round plate, it also extends externally toward all directions to get the object of the present invention.

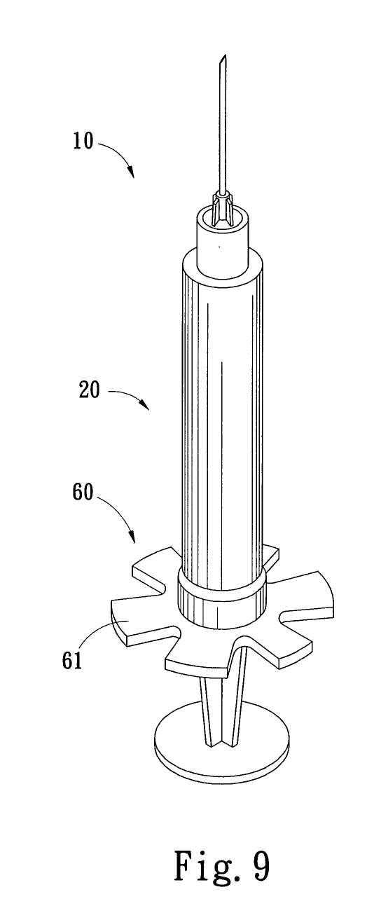

Referring to FIG. 9 showing a third embodiment of the present invention, wherein a bottom plate 60 on the rear end of the barrel 20 includes six wings 61 extending externally toward different directions and arranged in a circle equidistantly, the wings also extend externally toward about all directions and can also get rid of the conventional flaw of the direction limitation of wings.

The bottom plates 20, 50 and 60 of the barrel 20 in the present invention extend externally to different orientations, when the syringe 10 is placed on a desk, it will incline for an angle; however, there is a cover covering the needle set 30 before injection (not shown), the needle set 30 will be retractable into the barrel 20 after injection, hence there will be no such worry of the case stated above.

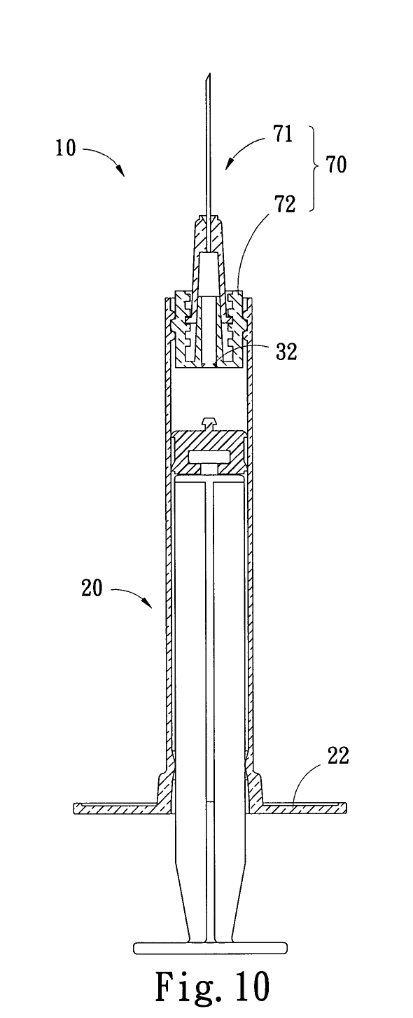

Referring to FIG. 10 showing another embodiment of the present invention, wherein the hexagonal bottom plate 22 can also suit a needle set 70 capable of being changed for a standard needle 71; the needle set 70 includes the standard needle 71 and an adapter 72, the standard needle 71 is detachably connected to the adapter 72 which looks like the rear end of the needle set 70, the abovementioned first engaging portion 32 is connected to the adapter 72; and the abovementioned different bottom plates 50 and 60 of the second and third engaging portions respectively are also suitable for using on the retractable safety type syringe 10.

Accordingly, the present invention has the following advantages:
1. By virtue that the bottom plate of a barrel extends externally toward all or nearly all directions, a medical staff can conveniently push or pull back the barrel with only single-hand.
2. If the medical staff is accustomed to inject or pull back the barrel with only single-hand, by the fact that fingers of each hand have predetermined lengths, the flaw of exerting overly large forces by two hands to pull the plunger and the needle set out of the barrel can be prevented.

As stated in the above disclosed conclusively, the present invention can surely achieve its expected objects to provide a retractable safety type syringe operatable with single-hand for a user for pushing in or drawing back a plunger in favor of drawing in or injecting liquid medicine. Having thus described my invention, what I claim as new and having practical value and desire to be secured by Letters Patent of the United States are:

1-8. (Canceled)

9. A retractable safety syringe operable with one hand comprising:
a) a barrel having a first barrel end and a bottom grip extending outwardly from an exterior of a second barrel end, the bottom grip is selected from a group consisting of a plate having a polygonal shaped outer periphery, a round plate having a rim, and at least three wings equally spaced around the second barrel end;
b) a needle set located on the first barrel end and having a first engaging portion; and
c) a plunger inserted into the barrel and having an end plate and a second engaging portion connected to the first engaging portion,
wherein the polygonal shaped outer periphery has a plurality of sides, each of the plurality of sides having an equal length.

10. The retractable safety syringe according to claim 9, wherein the bottom grip is the plate having the polygonal shaped outer periphery, and the polygonal shaped outer periphery has a rim.

11. The retractable safety syringe according to claim 9, wherein the bottom grip is the plate having the polygonal shaped outer periphery, and the polygonal shaped outer periphery of the bottom grip is a hexagonal.

12. The retractable safety syringe according to claim 11, wherein the polygonal shaped outer periphery has a rim.

13. The retractable safety syringe according to claim 9, wherein the bottom grip is the at least three wings, each of the at least three wings has a curved outer periphery with a common radius.

14. The retractable safety syringe according to claim 9, wherein the bottom grip is the at least three wings, and the at least three wings includes six wings.

15. The retractable safety syringe according to claim 14, wherein each of the six wings has a curved outer periphery with a common radius.

16. The retractable safety syringe according to claim 9, wherein the needle set includes an adapter and an injection needle removably connected to the adapter, the adapter is connected to the first engaging portion.
